(19) United States
(12) Patent Application Publication
(10) Pub. No.: US 2025/0277208 A1
(43) Pub. Date: Sep. 4, 2025

(54) Method of Peptide Library Construction and Peptides Thereof

(71) Applicant: Alcamena Stem Cell Therapeutics, LLC, Halethorpe, MD (US)

(72) Inventor: Alexander Pisarchik, Halethorpe, MD (US)

(21) Appl. No.: 18/987,348

(22) Filed: Dec. 19, 2024

Related U.S. Application Data
(60) Provisional application No. 63/559,346, filed on Feb. 29, 2024.

(51) Int. Cl. C12N 15/10; A61K 38/00; A61P 37/06; C07K 14/715; C12Q 1/34; C12Q 1/6855; G01N 33/68

(52) U.S. Cl. CPC C12N 15/1068; A61P 37/06; C07K 14/7155; C12N 15/1058; C12N 15/1093; C12Q 1/34; C12Q 1/6855; G01N 33/6869; A61K 38/00; G01N 2333/5412

(57) ABSTRACT

A method of library construction for peptide generation and, more particularly, to a method of construction of a DNA library built for selection of peptides that starts with a protein sequence known to bind to a target, wherein the protein sequence is used to generate DNA sequences which are then recombined and wherein each codon of the recombined DNA sequences has degenerate bases that cover between 1 and 20 of 20 possible amino acids.

### CROSS REFERENCE TO RELATED PATENT APPLICATIONS

This application claims priority to and the benefit of U.S. Provisional Patent Application No. 63/559,346, filed Feb. 29, 2024, which is incorporated by reference herein in its entirety for all purposes.

### SEQUENCE LISTING

The instant application contains a Sequence Listing which has been submitted electronically in XML file format and is hereby incorporated by reference in its entirety. Said XML copy, created on Dec. 18, 2024, is named ALC11366P00011US_SL.xml and is 139,911 bytes in size.

### BACKGROUND OF THE INVENTION

### Field of the Invention

The method of this disclosure relates to library construction for peptide generation and, more particularly, to construction of a DNA library built for selection of peptides that starts with a protein sequence known to bind to a target, wherein the protein sequence is used to generate DNA sequences which are then recombined and wherein each codon of the recombined DNA sequences has degenerate bases that cover between 1 and 20 of 20 possible amino acids.

### Description of Related Art

Directed evolution (DE) is one of the protein engineering approaches which relies on iterative cycles of DNA mutagenesis and selection/screening in order to isolate new proteins or peptides with improved function. Dougherty, M. J.; Arnold, F. H. Directed Evolution: New Parts and Optimized Function. Curr Opin Biotechnol 2009, 20 (4), 486-491. Diversity generation by various methods of peptide library construction is a critically important but is an undervalued step in any directed evolution experiment. The major challenge of diversity generation is that the total number of coding sequences that could be created is many orders of magnitude larger than what it is possible to create and test in a laboratory. Currin, A.; Parker, S.; Robinson, C. J.; Takano, E.; Scrutton, N. S.; Breitling, R. The Evolving Art of Creating Genetic Diversity: From Directed Evolution to Synthetic Biology, Biotechnol Adv 2021, 50, 107762. There is a wide variety of methods of diversity generation. Due to the simplicity of random peptide libraries, they remain the most commonly used libraries despite the fact that their large size makes them ineffective for most peptide screening approaches (e.g. phage and yeast display). For example, a random sequence for a 12 amino acid peptide generates more than 4×10²¹ sequences, while phage and yeast display can only handle 10¹⁰ sequences. Bazan, J.; Całkosiński, I.; Gamian, A. Phage Display—a Powerful Technique for Immunotherapy: 1. Introduction and Potential of Therapeutic Applications. Hum Vaccin Immunother 2012, 8 (12), 1817-1828. Even the most advanced methods like mRNA display cannot handle more than 10¹³ sequences. When using random peptide libraries shorter sequences, limited degeneracy, or other ways of controlling library size must be used.

In a more targeted “phylomer” approach, phylomer peptide libraries are built from genomic sequences. The expected success of these peptide libraries is based on the idea that peptides derived from stable, naturally selected proteins are more likely to develop the correct topography necessary for high-affinity binding to the target. Milech, N.; Watt, P. The Construction of “Phylomer” Peptide Libraries as a Rich Source of Potent Inhibitors of Protein/Protein Interactions. Methods Mol Biol 2012, 899, 43-60. However, Phylomer libraries have several limitations. First, they are constructed from the whole genome, which still results in a very large library. Second, they do not account for the fact that when removed from the context of the larger protein, peptides often lose their structure and activity. In order to preserve structure and function, peptides may need to be assembled from non-contiguous parts of the protein and may require additional mutations. Third, restoration of the structure of a peptide that has been removed from the context of a protein may require additional mutations, which is also not performed by the phylomer approach. What is needed is a library construction method for peptide generation which solves these problems.

A library construction method is disclosed herein that solves these problems. This method relies on the use of a protein known to bind the target. Residues that are crucial for ligand binding are identified and optimized through construction of the library followed by a selection. This method was used to develop an inhibitory peptide for IL-6, a multifunctional cytokine which plays a major role in many chronic inflammatory and lymphoproliferative disorders, including rheumatoid arthritis (RA), Crohn's disease and Castleman disease (CD). Because of its pathological role, IL-6 is an attractive target for the treatment of many debilitating diseases. However, currently marketed therapeutics targeting the IL-6 pathway have issues with long term tolerability and safety. For instance, the clinical use of antibodies against the IL-6R (currently the predominant therapeutic modality) can result in the development of neutralizing antibodies leading to treatment unresponsiveness and complications such as intestinal perforation (e.g., in patients with Crohn's disease or bacterial infections. Similarly, first generation antibodies against IL-6 used to treat multiple myeloma resulted in side effects including fever, fatigue, recurrent bone pain and hypercalcemia, due to antibodies keeping IL-6 in the circulation and leading to an increase of its blood level. Other drug modalities such as minibodies, single chain antibodies, DARPins, and avimers have also been tested, but their high cost, invasive administration, and a propensity for high immunogenicity may limit their clinical utility. Peptides, on the other hand, are a superior alternative to small molecules and biologics. Compared to small molecules, peptides exhibit high specificity and lower toxicity. Compared to antibodies, and biologics in general, they have better organ and tumor penetration, lower production cost and lower immunogenicity. However, the IL-6 targeting peptides described in the academic literature have not come to market.

Current phylomer libraries have three key flaws. First, they are constructed from the whole genome, which results in a very large library. Second, they do not account for the fact that when removed from the context of the larger protein, peptides often lose their structure and activity. In order to preserve their structure, the peptides may need to be assembled from non-contiguous parts of the protein, which is not performed in the phylomer approach. Third, restoration of the structure of a peptide that has been removed from the context of a protein may require additional mutations, which is also not performed by the phylomer approach. What is needed is a library construction method for peptide generation which solves these problems.

### BRIEF SUMMARY OF THE INVENTION

Disclosed herein is a method of constructing a peptide library comprising:
1) identifying a protein of interest;
2) synthesizing DNA primers comprising DNA sequences, wherein the DNA sequences are determined by the following steps:
a) writing a DNA sequence that codes for the identified protein or a segment thereof;
b) splitting the written DNA sequence of the identified protein or segment thereof into a plurality of written DNA sequences from 3 to 18 nucleotides or longer, thereby forming a list of short written DNA sequences;
c) introducing degeneracy into one or more of the short written DNA sequence from step (b) to create one or more short degenerate written DNA sequences; and
d) adding the one or more short degenerate written DNA sequences to written DNA primer sequences designed to amplify a DNA fragment of a screening construct to create written DNA primer sequences with overhangs; and
e) synthesizing the written DNA primer sequences with overhangs from step (d) to create synthesized primers;
3) amplifying DNA fragments using at least three or more of the synthesized primers from step (e) and one or more DNA polymerases;
4) mixing, phosphorylating and ligating the amplified DNA fragments from step (3), thereby forming a DNA library; and
5) expressing peptides coded by the DNA library formed in step (4) in an appropriate system in vitro or in vivo, thereby forming a first peptide library having peptides with a pharmacologic activity.

In a preferred embodiment, in step (c), degeneracy is introduced into the one or more short written DNA sequences by one of the following steps:
changing the nucleotide at the first position of every codon;
changing the nucleotide at the second position of every codon;
changing the nucleotide at the first position of the first codon, the second position of the second codon and repeating this pattern for the entire short written DNA sequence;
changing the second position of the first codon and the first position of the second codon and repeating this pattern for the entire short written DNA sequence; and
changing the first position and/or the second position of less than every codon.

In a preferred embodiment, steps (2) through (5) are repeated, thereby, forming a second peptide library with an improved pharmacologic activity, compared to the first peptide library.

In another embodiment, the segment of the identified protein consists of loops and/or secondary protein structures.

In one embodiment, the size of the binding domain is at least 220 or more amino acids, preferably 250 or more amino acids.

In one embodiment, in step 3, the primers are used simultaneously.

In one embodiment, in step 3, one or more of the synthesized primers are designed for a segment of the binding domain, wherein the segment comprises 10 or more amino acids.

In one embodiment, in step 3, one or more, or two or more, or all of the synthesized primers comprises at least 30 nucleotides, and preferably, at least 60 nucleotides.

In one embodiment, amplified DNA fragments from step 3 are digested by type IIS restriction enzymes.

In one embodiment, ligated DNA fragments from step 4 are amplified with the flanking primers.

In one embodiment, each DNA sequence in step (c) is different from the DNA sequence from step (b) by only one codon.

In one embodiment, the degenerate written short DNA sequences in step 3 are linked as overhangs to primers with one forward primer and one reverse primer.

In one embodiment, the one or more DNA polymerases in step (e) comprise a Vent polymerase generating blunt DNA ends.

In one embodiment, the methods of the invention further comprise the following steps:
6) selecting and synthesizing peptides from the first or second peptide library;
7) measuring an affinity of binding of the selected and synthesized peptides from the first or second library to the desired biological target; and
8) measuring efficacy of the selected and synthesized peptides from the first or second library in vitro and/or in vivo.

In one embodiment, residues that are crucial for ligand binding are identified and optimized through construction of the library followed by a selection. This method was used to develop a neutralizing peptide for IL-6, a multifunctional cytokine which plays a major role in many chronic inflammatory and lymphoproliferative disorders, including rheumatoid arthritis (RA), Crohn's disease and Castleman disease (CD).

In one embodiment, the invention provides a peptide from a peptide library produced by the methods of the invention.

In one embodiment, the invention provides a peptide from a peptide library produced by the methods of the invention, the peptide comprising the amino acid sequence as set forth in any one of SEQ ID Nos: 34, 35, 54, 55, 70, 73, and 83, or an amido acid sequence having more than 70%, 75%, 80%, 85%, 90% or 95% homology thereto.

In one embodiment, the peptide termini are protected.

In one embodiment, the protection is done by making the N terminus of the peptide acetylated and by making the C terminus amidated.

In a preferred embodiment, the peptide is SEQ ID No: 55 or an amido acid sequence having more than 70%, 75%, 80%, 85%, 90% or 95% homology thereto.

In one embodiment, the invention provides a method of inhibiting IL-6 signaling in a cell, the method comprising contacting a cell with an effective amount of the peptide of the invention.

In a preferred embodiment, the peptide is one of SEQ ID Nos: 34, 35, 54, 55, 70, 73, and 83, or an amido acid sequence having more than 70%, 75%, 80%, 85%, 90% or 95% homology thereto.

In an even more preferred embodiment, the peptide is SEQ ID No: 55 or an amido acid sequence having more than 70%, 75%, 80%, 85%, 90% or 95% homology thereto.

In one embodiment, the invention provides a method of treating a disease or condition mediated by IL-6 mechanism comprising administering to a patient in need thereof an effective amount of the peptide of the invention.

In one embodiment, the disease or condition comprises an inflammatory, degenerative or an autoimmune disease or condition.

In one embodiment, the disease or condition is selected from the group consisting of rheumatoid arthritis, Crohn's disease, Castleman disease, systemic lupus erythematosus, juvenile idiopathic arthritis, giant cell arteritis, ulcerative colitis, psoriatic arthritis, ankylosing spondylitis, multiple myeloma, systemic sclerosis, Still's disease, severe acute respiratory syndrome coronavirus 2 (SARS-CoV-2) infection (COVID-19), chronic obstructive pulmonary disease, atherosclerosis, osteoporosis, type 2 diabetes mellitus, depression, Alzheimer's disease, and cytokine release syndrome.

### BRIEF DESCRIPTION OF THE DRAWINGS

FIG. 1 is a flow chart of a method for generating pharmacologically active peptides based upon a library constructed of DNA sequences.

FIG. 2 is a flow chart showing steps of a method for discovering pharmacologically active peptide leads from the generation of pharmacologically active peptides based upon the DNA sequences (peptide library).

FIG. 3 is a flow chart illustrating a method of DNA library construction.

FIG. 4 illustrates alignment of IL-6R and peptide 9 sequences.

FIG. 5 shows a method of constructing an optimization library to improve peptide affinity.

FIG. 6 shows an alignment of peptide leads generated as described by affinity maturation.

FIG. 7 shows a graph illustrating inhibition of IL-6-induced cell signaling by selected peptides.

### DETAILED DESCRIPTION OF THE INVENTION

A library construction method is disclosed herein that solves the problems described above and relies on the use of a protein ligand known to bind to a biological target of intertest. Peptide residues that are crucial for binding of the protein ligand to the biological target can be identified and optimized through construction of a specialized library for generating peptides, followed by a selection of peptides that bind sufficiently to the protein target.

The efficacy of the method of library construction and active peptide selection described herein is demonstrated by the resulting identification of a highly pharmacologically active peptide.

In one embodiment, the peptide exhibits a high binding affinity for interleukin-6 (IL-6) and effectively inhibits the biological function of IL-6.

Thus, in one embodiment, the invention provides a method of constructing a peptide library comprising:
1) identifying a protein of interest;
2) synthesizing DNA primers comprising DNA sequences, wherein the DNA sequences are determined by the following steps:
a) writing a DNA sequence that codes for the identified protein or a segment thereof;
b) splitting the written DNA sequence of the identified protein or segment thereof into a plurality of written DNA sequences from 3 to 18 nucleotides or longer, thereby forming a list of short written DNA sequences;
c) introducing degeneracy into one or more of the short written DNA sequence from step (b) to create one or more short degenerate written DNA sequences; and
d) adding the one or more short degenerate written DNA sequences to written DNA primer sequences designed to amplify a DNA fragment of a screening construct to create written DNA primer sequences with overhangs; and
e) synthesizing the written DNA primer sequences with overhangs from step (d) to create synthesized primers;
3) amplifying DNA fragments using at least three or more of the synthesized primers from step (e) and one or more DNA polymerases;
4) mixing, phosphorylating and ligating the amplified DNA fragments from step (3), thereby forming a DNA library; and
5) expressing peptides coded by the DNA library formed in step (4) in an appropriate system in vitro or in vivo, thereby forming a first peptide library having peptides with a pharmacologic activity.

In a preferred embodiment, in step (c), degeneracy is introduced into the one or more short written DNA sequences by one of the following steps:
changing the nucleotide at the first position of every codon;
changing the nucleotide at the second position of every codon;
changing the nucleotide at the first position of the first codon, the second position of the second codon and repeating this pattern for the entire short written DNA sequence;
changing the second position of the first codon and the first position of the second codon and repeating this pattern for the entire short written DNA sequence; and
changing the first position and/or the second position of less than every codon.

In a preferred embodiment, steps (2) through (5) are repeated, thereby, forming a second peptide library with an improved pharmacologic activity, compared to the first peptide library.

In another embodiment, the segment of the identified protein consists of loops and/or secondary protein structures.

In one embodiment, the size of the binding domain is at least 220 or more amino acids, preferably 250 or more amino acids.

In one embodiment, in step 3, the primers are used simultaneously.

In one embodiment, in step 3, one or more of the synthesized primers are designed for a segment of the binding domain, wherein the segment comprises 10 or more amino acids.

In one embodiment, in step 3, one or more, or two or more, or all of the synthesized primers comprises at least 30 nucleotides, and preferably, at least 60 nucleotides.

In one embodiment, amplified DNA fragments from step 3 are digested by type IIS restriction enzymes.

In one embodiment, ligated DNA fragments from step 4 are amplified with the flanking primers.

In one embodiment, each DNA sequence in step (c) is different from the DNA sequence from step (b) by only one codon.

In one embodiment, the degenerate written short DNA sequences in step 3 are linked as overhangs to primers with one forward primer and one reverse primer.

In one embodiment, the one or more DNA polymerases in step (e) comprise a Vent polymerase generating blunt DNA ends.

In one embodiment, the methods of the invention further comprise the following steps:
6) selecting and synthesizing peptides from the first or second peptide library;
7) measuring an affinity of binding of the selected and synthesized peptides from the first or second library to the desired biological target; and
8) measuring efficacy of the selected and synthesized peptides from the first or second library in vitro and/or in vivo.

In one embodiment, residues that are crucial for ligand binding are identified and optimized through construction of the library followed by a selection. This method was used to develop a neutralizing peptide for IL-6, a multifunctional cytokine which plays a major role in many chronic inflammatory and lymphoproliferative disorders, including rheumatoid arthritis (RA), Crohn's disease and Castleman disease (CD).

In one embodiment, the invention provides a peptide from a peptide library produced by the methods of the invention.

In one embodiment, the invention provides a peptide from a peptide library produced by the methods of the invention, the peptide comprising the amino acid sequence as set forth in any one of SEQ ID Nos: 34, 35, 54, 55, 70, 73, and 83, or an amido acid sequence having more than 70%, 75%, 80%, 85%, 90% or 95% homology thereto.

In one embodiment, the peptide termini are protected.

In one embodiment, the protection is done by making the N terminus of the peptide acetylated and by making the C terminus amidated.

In a preferred embodiment, the peptide is SEQ ID No: 55 or an amido acid sequence having more than 70%, 75%, 80%, 85%, 90% or 95% homology thereto.

In one embodiment, the invention provides a method of inhibiting IL-6 signaling in a cell, the method comprising contacting a cell with an effective amount of the peptide of the invention.

In a preferred embodiment, the peptide is one of SEQ ID Nos: 34, 35, 54, 55, 70, 73, and 83, or an amido acid sequence having more than 70%, 75%, 80%, 85%, 90% or 95% homology thereto.

In an even more preferred embodiment, the peptide is SEQ ID No: 55 or an amido acid sequence having more than 70%, 75%, 80%, 85%, 90% or 95% homology thereto.

In one embodiment, the invention provides a method of treating a disease or condition mediated by IL-6 mechanism comprising administering to a patient in need thereof an effective amount of the peptide of the invention.

In one embodiment, the disease or condition comprises an inflammatory, degenerative or an autoimmune disease or condition.

In one embodiment, the disease or condition is selected from the group consisting of rheumatoid arthritis, Crohn's disease, Castleman disease, systemic lupus erythematosus, juvenile idiopathic arthritis, giant cell arteritis, ulcerative colitis, psoriatic arthritis, ankylosing spondylitis, multiple myeloma, systemic sclerosis, Still's disease, severe acute respiratory syndrome coronavirus 2 (SARS-CoV-2) infection (COVID-19), chronic obstructive pulmonary disease, atherosclerosis, osteoporosis, type 2 diabetes mellitus, depression, Alzheimer's disease, and cytokine release syndrome.

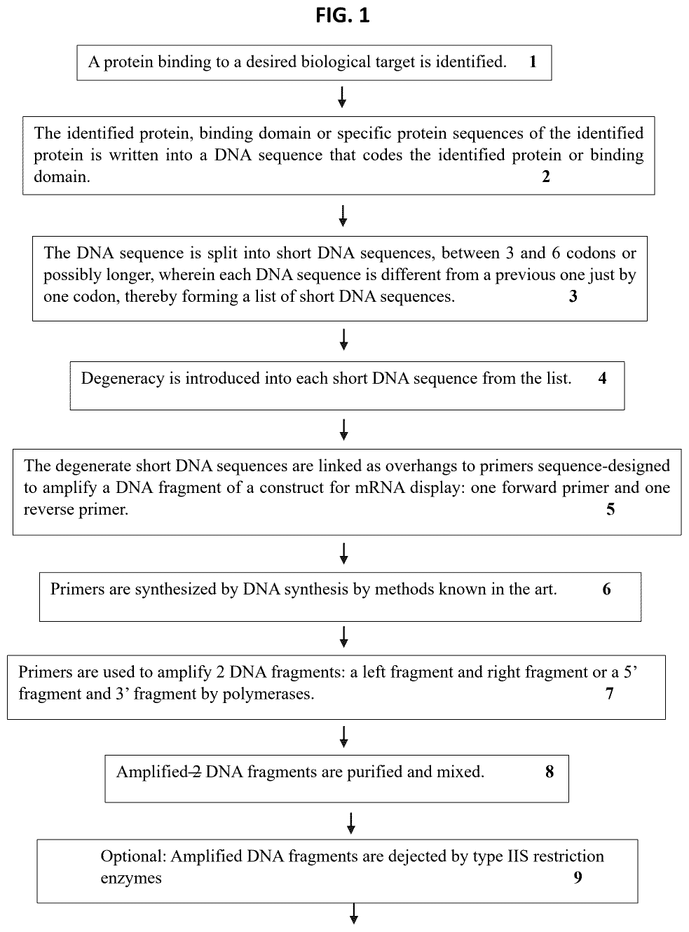

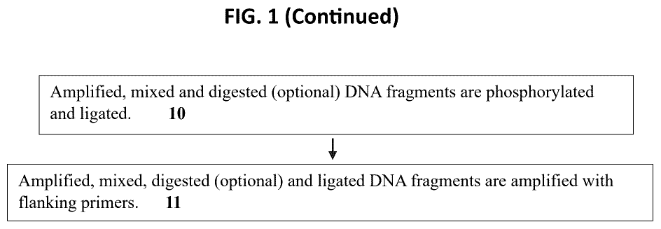

FIG. 1 is a flow chart of a method for generating pharmacologically active peptides based upon a library constructed of DNA sequences. A protein that binds to a desired biological target is identified (Step 1). The identified protein or a binding domain peptide sequence of the identified protein is written into a DNA sequence that codes the identified protein or binding domain peptide sequence (Step 2). The DNA sequence is split into short DNA sequences, between 3 and 6 codons or possibly longer, wherein each DNA sequence is different from a previous one just by one codon, thereby forming a list of short DNA sequences (Step 3). For example, the list may provide Option A—2 codons (6 bp), Option B—3 codons (9 bp), Option C—4 codons (12 bp), Option D—5 codons (15 bp), Option E—6 codons (18 bp), Option F—more than 6 codons, wherein bp is base pairs.

Degeneracy is introduced into each short DNA sequence from the list (Step 4). For example, Option A—first position of each codon is mutated; Option B—second position of each codon is mutated; Option C—the first position of the first codons is mutated; the second position of the second codon is mutated and so on; Option D—the second position of the first codons is mutated; the first position of the second codon is mutated and so on; Option E—only some codons are mutated; and Option F—other known types of degenerate codons are used. The degenerate short DNA sequences are linked as overhangs to primers sequence-designed to amplify a DNA fragment of a construct for screening or selection: one forward primer and one reverse primer (Step 5). For Option A—Primers have sequence annealing to a selection construct and overhang with degenerate codons only. These primers are used to generate DNA fragments with blunt ends. For Option B—Primers have sequence annealing to the selection construct, overhang with degenerate codons and additional sequence recognized by type IIS restriction enzymes (that cut outside their recognition sequence). These primers are used to generate DNA fragments with sticky ends.

Primers are synthesized by DNA synthesis by methods known in the art (Step 6). Primers are used to amplify 2 DNA fragments: a left fragment and right fragment (or a 5′ fragment and 3′ fragment) by Vent polymerase which generates blunt DNA ends (Step 7). Amplified DNA fragments are purified and mixed (Step 8). Amplified DNA fragments are dejected by type II restriction enzymes (if additional sequence recognized by type IIS restriction enzymes are attached) (Step 9). Amplified and digested (optional step) DNA fragments are phosphorylated and ligated (Step 10). Amplified, digested (optional step) and ligated DNA fragments are amplified with flanking primers (Step 11).

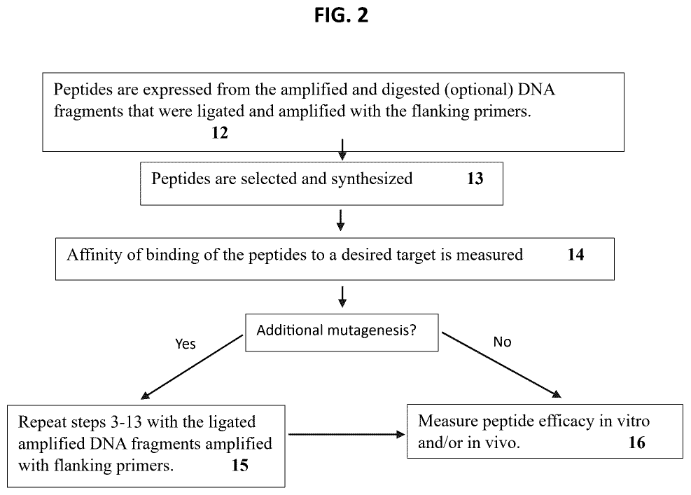

FIG. 2 is a flow chart showing steps of a method for discovering pharmacologically active peptide leads from the generation of pharmacologically active peptides based upon the DNA sequences (peptide library). DNA fragments that were ligated and amplified with the flanking primers are used to express peptides which are then used for screening or selection (Step 12). Peptides that perform the best are selected from screening and selection and synthesized (Step 13). Affinity of binding of the peptides to a desired target is measured (Step 14). Additional mutagenesis may be desired. If so, steps 3-13 are repeated with the DNA fragments from step 12 (Step 15). If not, peptide pharmacologic efficacy is measured in vitro and/or in vivo (Step 16).

### Comparison of PepFusion Libraries to Other Methods

Making biologically active peptides out of existing proteins has so far been challenging. First, when peptides are isolated from larger proteins, they tend to lose their secondary structure and activity. To preserve secondary structure and function, mutations may be need to be introduced in the peptide sequence. However, there is currently no way to predict the required mutations. Second, residues involved in protein-protein interactions may come from specially separated non-contiguous parts of the protein so insertions and deletions may need to be introduced in the final peptide. Again, the location and size of these insertions and deletions is impossible to predict. Due to this enormous uncertainty, there is currently no method that can reliably create functional peptides from proteins. PepFusion is the first method to overcome these challenges.

Because the claimed method of library construction tackles several problems simultaneously, it is hard to compare it to the existing methods. It involves mutagenesis but unlike error-prone PCR or in vivo mutagenesis, it introduces degeneracy through synthetic primers which makes it closer to synthetic shuffling. But unlike synthetic shuffling or other shuffling approaches, the inventive method does not require DNA homology. Similar to Nonhomologous Random Recombination (NRR), it uses ligation to connect different DNA fragments, but unlike NRR, it ligates two fragments only.

Additionally, the invention's approach does not introduce frame-shifts and is not as random as NRR. Even though reengineering the whole protein is possible, in a preferred embodiment, primers are designed to the specialized parts or the protein, such as substrate- or protein-binding domains or the specific loops involved in protein interaction. Therefore, PepFusion library construction method combines mutagenesis and recombination and accelerates peptide drug discovery by simultaneous identification and optimization of the residues most critical for protein-protein interactions and ligand binding.

Another potential feature of the PepFusion libraries lies in the ability to discriminate specific and non-specific binders. As is well known in the field, in addition to specific binders, modern display technologies often yield a variety of non-specific binders with no affinity toward the target. These sequences are recovered due to their propagation advantages or binding to other components of the screening system (such as the solid phase, capturing reagents, blocking agents), rather than the target. The PepFusion library makes it possible to eliminate false positives at the stage of data analysis by simple comparison to the DNA sequence of the parent gene.

The invention allows using either linear peptides or cyclic peptides.

In one embodiment, linear peptides are used to construct a peptide library.

In another embodiment, cyclic peptides are used to construct a peptide library, wherein flanking cysteines are added, as is known in the art.

The following Examples are intended to illustrate further certain embodiments of the invention and are not intended to limit the scope of the invention.

### EXAMPLES OF THE INVENTION

### Example 1

As an example of using the methods of this disclosure a peptide library was constructed from the Interleukin-6 Receptor (IL-6R) sequence that binds the protein Interleukin-6 (IL-6). Building a library out of naturally selected protein increases the chances of finding a peptide with the correct amino acid sequence and topography necessary for high-affinity binding.

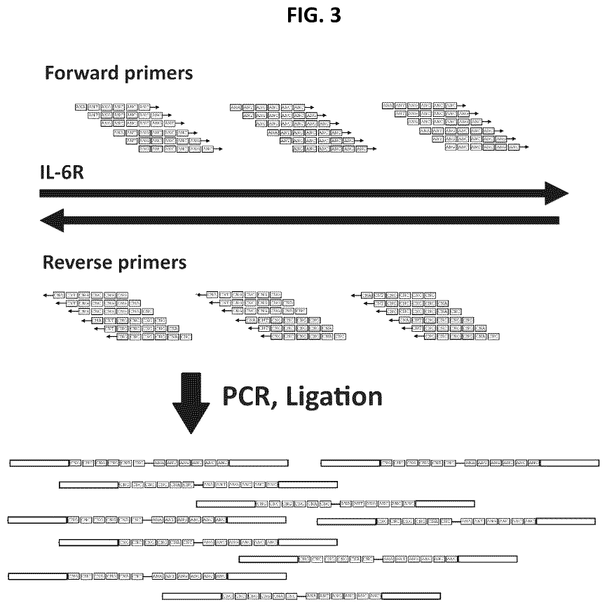

The method of DNA library construction is presented in FIG. 3. Two DNA fragments were amplified from a synthetic fragment of the IL-6R. The 5′ fragment was amplified with forward primer and a mixture of 432 reverse primers, each containing an overhang of 6 codons. The 3′ fragment was amplified with a mixture of 432 forward primers, also containing overhangs of six degenerate codons and the reverse primer. Either the first or the second nucleotide of each codon was replaced with a degenerate base. Following PCR amplification, DNA fragments were phosphorylated and ligated leading to various sequence rearrangements shown at the bottom of the FIG. 3. Four hundred thirty-two forward and reverse oligos with an overhang of 6 codons from the IL-6R binding domain were used for PCR. A degenerate nucleotide was inserted at the first or second position of each codon. When ligated, these sequences generated a library of 1×10¹³ different DNA sequences. The library of DNA sequences was taken through eight rounds of mRNA Display generating a peptide library. Eighteen of the most abundant peptides in the peptide library were synthesized in a linear form and tested for binding to IL-6 using Biacore. One of the peptides (#9) (“Peptide 9”) from the peptide library, with the sequence of SVRDLLRRMCHIVG (SEQ ID NO: 1), was confirmed to bind IL-6 with a KD of 2.5 μM. The twelve non-terminal residues represent the PepFusion library-derived DNA sequences, while N-terminal serine and C-terminal glycine come from the flanking linkers and were kept as a precaution, in case linker sequences contributed to peptide interaction with its target.

To test the utility of the peptide library, it was compared to a random library, generated with 12 degenerate codons, resulting in up to 4×10²¹ sequences. This random library yielded no peptides with measurable binding affinity.

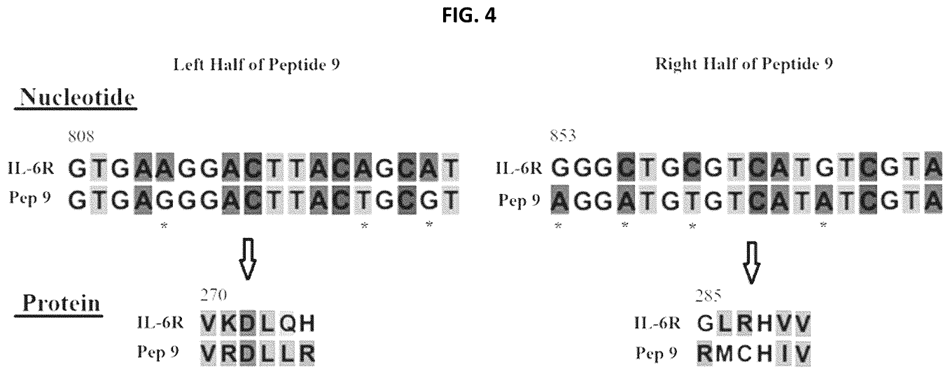

Analysis of Peptide 9 revealed that Peptide 9 comprised two distinct segments originating from separate regions of the IL-6 receptor. Nucleotide alignment showed that the N-terminal segment is derived from residues 270 to 275 of the receptor, while the C-terminal segment comes from residues 285 to 290 as shown in FIG. 4. FIG. 4 shows alignment of IL-6R and Peptide 9 sequences. The top panel shows the alignment of the IL-6 receptor and peptide 9 nucleotide sequences. The bottom panel shows protein alignment. Numbers correspond either to nucleotide (top panel) or amino acid positions (bottom panel) of the full-length IL-6R. Asterisks mark the nucleotide substitutions. Both of these regions of the IL-6R were previously recognized as crucial for IL-6 binding. Three codons from the N-terminal part of the sequence have mutations at the second nucleotides, while the C-terminal part has 4 mutations located in the first position of each codon, which is consistent with our DNA library design described above (FIG. 3).

### Example 2

### Affinity Maturation

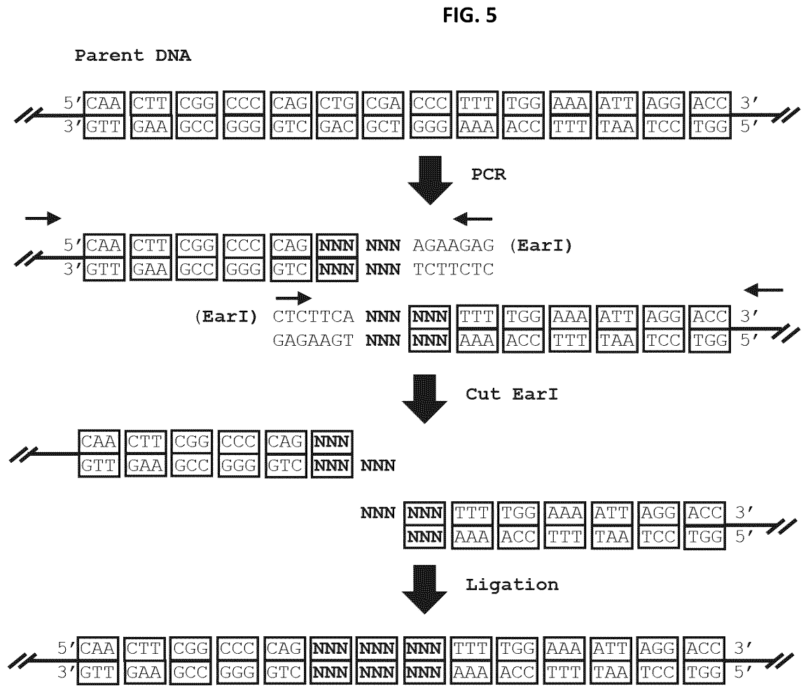

An additional round of mutagenesis and peptide selection was performed on the library of DNA sequences in order to improve peptide 9 affinity. FIG. 5 shows a method of constructing an optimization library. Two DNA fragments were amplified from the synthetic DNA fragment.

The 5′ fragment was amplified with the forward primer and a reverse primer containing an overhang of degenerate codons and an EarI restriction site. The 3′ fragment was amplified with a forward primer containing an overhang of degenerate codons and an EarI restriction site, and the reverse primer. Following amplification, DNA fragments were cut with EarI and ligated leading to the final construct with three degenerate codons. The peptide construct was mutagenized as described in FIG. 3 with a few differences: each of the forward and reverse oligos had only one degenerate codon and the ligation was performed through the overhangs generated by the EarI restriction enzyme. Upon PCR, digestion with EarI and ligation, a set of DNA libraries was generated, each containing 3 degenerate codons. After one round of mRNA display the 30 most abundant peptide sequences were synthesized and assessed for binding.

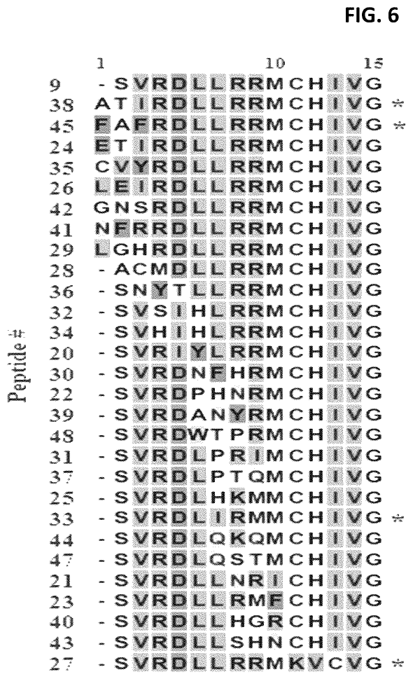

FIG. 6 shows in a column the alignment of the peptide leads generated as described by affinity maturation. Parent peptide 9 is shown at the top of the column. Peptide numbers are shown on the left of the column. Residue position numbers are shown at the top of the column and correspond to the residue positions. Peptides with the best affinity are marked with an asterisk. As illustrated in the sequence alignment of the selected peptides mutations were located through the whole sequence. Binding data showed that the majority of the tested variants did not have any improvements in binding affinity (Table 1).

TABLE 1
Affinity of the peptides picked from the
affinity maturation library. N and C termini
are not protected

| Peptide | | KD (nM) | |
|---|---|---|---|
| Number | Sequence | P < 0.05 | Seq. ID |
| 9 | -SVRDLLRRMCHIVG | 2585 ± 176 | SEQ ID NO: 1 |
| | (parent) | | |
| 20 | -SVRIYLRRMCHIVG | >10 μM | SEQ ID NO: 2 |
| 21 | -SVRDLLNRICHIVG | >10 μM | SEQ ID NO: 3 |
| 22 | -SVRDPHNRMCHIVG | >10 μM | SEQ ID NO: 4 |
| 23 | -SVRDLLRMFCHIVG | >10 μM | SEQ ID NO: 5 |
| 24 | ETIRDLLRRMCHIVG | 119 ± 11 | SEQ ID NO: 6 |
| 25 | -SVRDLHKMMCHIVG | >10 μM | SEQ ID NO: 7 |
| 26 | LEIRDLLRRMCHIVG | >10 μM | SEQ ID NO: 8 |
| 27 | -SVRDLLRRMKVCVG | 58 ± 6 | SEQ ID NO: 9 |
| 28 | -ACMDLLRRMCHIVG | 2730 | SEQ ID NO: 10 |
| 29 | LGHRDLLRRMCHIVG | >10 μM | SEQ ID NO: 11 |
| 30 | -SVRDNFHRMCHIVG | >10 μM | SEQ ID NO: 12 |
| 31 | -SVRDLPRIMCHIVG | >10 μM | SEQ ID NO: 13 |
| 32 | -SVSIHLRRMCHIVG | >10 μM | SEQ ID NO: 14 |
| 33 | -SVRDLIRMMCHIVG | 545 ± 13 | SEQ ID NO: 15 |
| 34 | -SVHIHLRRMCHIVG | >10 μM | SEQ ID NO: 16 |
| 35 | CVYRDLLRRMCHIVG | nd | SEQ ID NO: 17 |
| 36 | -SNYTLLRRMCHIVG | >10 μM | SEQ ID NO: 18 |
| 37 | -SVRDLPTQMCHIVG | 649 ± 25 | SEQ ID NO: 19 |
| 38 | ATIRDLLRRMCHIVG | 54 ± 8 | SEQ ID NO: 20 |
| 39 | -SVRDANYRMCHIVG | nd | SEQ ID NO: 21 |
| 40 | -SVRDLLHGRCHIVG | >10 μM | SEQ ID NO: 22 |
| 41 | NERRDLLRRMCHIVG | >10 μM | SEQ ID NO: 23 |
| 42 | GNSRDLLRRMCHIVG | >10 μM | SEQ ID NO: 24 |
| 43 | -SVRDLLSHNCHIVG | nd | SEQ ID NO: 25 |
| 44 | -SVRDLQKOMCHIVG | nd | SEQ ID NO: 26 |
| 45 | FAFRDLLRRMCHIVG | 92 ± 11 | SEQ ID NO: 27 |
| 46 | SVRDLLRRMCHPRLG | >10 μM | SEQ ID NO: 28 |
| 47 | -SVRDLQSTMCHIVG | nd | SEQ ID NO: 29 |
| 48 | -SVRDWTPRMCHIVG | 2820 | SEQ ID NO: 30 |
| 49 | -SVRDLLRRMCVFWG | nd | SEQ ID NO: 31 |

nd-not detected

A large number of peptides completely lost binding affinity (peptides 35, 39, 43, 44, 47 and 49) or had reduced binding affinity (peptides 20, 21, 22, 23, 25, 26, 29, 31, 34, 36, 40, 41, 42, 46). However, there were several peptides with improved binding affinity (Table 1, peptides 24, 27, 33, 38 and 45). Two of them (peptides 38 and 45) had three mutations located at the N terminus of the peptide, implying that this region might be a key site of interaction between the peptide and IL-6.

### Example 3

### Combinatorial Mutagenesis

Mutations were assessed for their contribution to affinity (KD) for IL-6. Several peptides were synthesized with either individual mutations or their combinations and compared the resulting affinities (Table 2). The peptide termini were protected with acetylation and amination to make them resistant to proteases.

TABLE 2
Affinity of the peptide variants with different mutation combinations
N terminus is acetylated, C terminus is amidated

| | | KD (nM) | |
|---|---|---|---|
| Peptide Number | Sequence | P < 0.05 | Seq ID |
| 9(2) | 1 5 10 15 | 354 ± 69 | SEQ ID NO: 32 |
| | -SVRDLLRRMCHIVG | | |
| 27(2) | -SVRDLLRRMKVCVG | 580 ± 156 | SEQ ID NO: 33 |
| 38(2) | ATIRDLLRRMCHIVG | 115 ± 33 | SEQ ID NO: 34 |
| 45(2) | FAFRDLLRRMCHIVG | 147 ± 31 | SEQ ID NO: 35 |
| 51 | ATIRDLIRMMCHIVG | 128 ± 7 | SEQ ID NO: 36 |
| 52 | ATIRDLLSHNCHIVG | nd | SEQ ID NO: 37 |
| 53 | ATIRDLLRRMKVCVG | 436 ± 21 | SEQ ID NO: 38 |
| 54 | ASVRDLLRRMCHIVG | 422 ± 17 | SEQ ID NO: 39 |
| 55 | -TVRDLLRRMCHIVG | 224 ± 11 | SEQ ID NO: 40 |
| 56 | -SIRDLLRRMCHIVG | 305 ± 11 | SEQ ID NO: 41 |
| 58 | FAFRDLIRMMCHIVG | 71 ± 7 | SEQ ID NO: 42 |
| 59 | FAFRDLLSHNCHIVG | nd | SEQ ID NO: 43 |
| 60 | FAFRDLLRRMKVCVG | nd | SEQ ID NO: 44 |
| 61 | FSVRDLLRRMCHIVG | 144 ± 10 | SEQ ID NO: 45 |
| 62 | -AVRDLLRRMCHIVG | 519 ± 18 | SEQ ID NO: 46 |
| 63 | -SFRDLLRRMCHIVG | 162 ± 14 | SEQ ID NO: 47 |
| 64 | -SVRDLIRMMKVCVG | 1390 ± 42 | SEQ ID NO: 48 |
| 65 | -SVRDLLRRMKHIVG | 150 ± 15 | SEQ ID NO: 49 |
| 66 | -SVRDLLRRMCVIVG | 283 ± 13 | SEQ ID NO: 50 |
| 67 | -SVRDLLRRMCHCVG | nd | SEQ ID NO: 51 |
| 68 | -SVRDLIRRMCHIVG | 724 ± 28 | SEQ ID NO: 52 |
| 69 | -SVRDLLRMMCHIVG | 172 ± 11 | SEQ ID NO: 53 |
| 71 | -TIRDLLRRMCHIVG | 49 ± 40 | SEQ ID NO: 54 |
| 72 | FSFRDLLRRMCHIVG | 27 ± 20 | SEQ ID NO: 55 |
| 73 | -SVRDLLRRMKVIVG | 1133 ± 18 | SEQ ID NO: 56 |
| 74 | FSFRDLIRMMCHIVG | >10 μM | SEQ ID NO: 57 |
| 75 | FAFRDLLRMMCHIVG | 132 ± 6 | SEQ ID NO: 58 |
| 76 | FAFRDLIRMMKVIVG | 1300 ± 14 | SEQ ID NO: 59 |
| 77 | FAFRDLIRMMKHIVG | 405 ± 15 | SEQ ID NO: 60 |
| 78 | FAFRDLIRMMCVIVG | 1486 ± 22 | SEQ ID NO: 61 |
| 79 | FAFRDLIRMMKVCVG | 247 ± 13 | SEQ ID NO: 62 |
| 80 | FSFRDLLRMMCHIVG | 249 ± 11 | SEQ ID NO: 63 |
| 81 | FSFRDLLRMMKVIVG | 1272 ± 17 | SEQ ID NO: 64 |
| 82 | ATIRDLIRMMKVCVG | 108 ± 83 | SEQ ID NO: 65 |
| 83 | ATIRDLIRMMKVIVG | 563 ± 23 | SEQ ID NO: 66 |
| 84 | -TIRDLLRMMKVIVG | 565 ± 18 | SEQ ID NO: 67 |
| 85 | FSVRDLLRRMKHIVG | >10 μM | SEQ ID NO: 68 |
| 86 | FSVRDLLRMMKHIVG | >10 μM | SEQ ID NO: 69 |
| 87 | FSVRDLIRMMKVCVG | 47 ± 11 | SEQ ID NO: 70 |
| 88 | -SFRDLLRRMKHIVG | >10 μM | SEQ ID NO: 71 |
| 89 | -SFRDLLRMMKHIVG | >10 μM | SEQ ID NO: 72 |
| 90 | -SFRDLIRMMKVCVG | 42 ± 17 | SEQ ID NO: 73 |
| 91 | FSFRDLLRRMKHIVG | >10 μM | SEQ ID NO: 74 |
| 92 | FSFRDLLRMMKHIVG | >10 μM | SEQ ID NO: 75 |
| 93 | FSFRDLIRMMKVCVG | 265 ± 11 | SEQ ID NO: 76 |
| 94 | ATIRDLLRRMKHIVG | >10 μM | SEQ ID NO: 77 |
| 95 | ATIRDLLRMMKHIVG | >10 μM | SEQ ID NO: 78 |
| 96 | ATIRDLIRMMKHIVG | >10 μM | SEQ ID NO: 79 |
| 97 | -TIRDLLRRMKHIVG | >10 μM | SEQ ID NO: 80 |
| 98 | -TIRDLLRMMKHIVG | >10 μM | SEQ ID NO: 81 |
| 99 | -TIRDLIRMMKHIVG | >10 μM | SEQ ID NO: 82 |
| 100 | -TIRDLIRMMKVCVG | 59 ± 12 | SEQ ID NO: 83 |

Control Peptides

| | | | |
|---|---|---|---|
| CA11 | NQQLIEEIIQILHKIFEIL | 50750 ± 35 | SEQ ID NO: 84 |
| | | 3 | |
| RA07 | INTLLSEINSILLDIISLL | nd | SEQ ID NO: 85 |
| PN-2519 | FD(Cha)DIHLL(4F)LPTEWEK(Cla)KNEE | nd | SEQ ID NO: 86 |
| PN-1974 | FD(hL)DIHLLFLPTEWEKDKNEE | 61 ± 11 | SEQ ID NO: 87 |
| PN-1974(2) | FD(hL)DIHLLELPTEWEKDKNEE | nd | SEQ ID NO: 88 |
| | N terminus acetylated | | |
| | C terminus amidated | | |
| C1 | -SVKDLQHGLRHVVG | nd | SEQ ID NO: 89 |

nd-not detected
(hL)-homoleucine
(Cha)-cyclohexyl-L-alanine
(Cla)-gamma-carboxyglutamic acid
(4F)-4fluoro-L-phenylalanine

The binding data showed that termini protection of the peptide 9 improved its KD from 2.5 μM to 300 nM. Similar improvements were observed for peptides 27, 38, and 45, which was also attributed to termini protection (Table 2). Analysis of individual mutations in peptide 38 (Table 2, peptides 38 (2), 54, 55 and 56), did not reveal which mutations contributed the most to the affinity improvement. In fact, the affinity of variants with individual mutations was similar to that of the parent sequence (peptide 9). However, when two out of the three mutations (S2T and V3I, Table 2, peptide 71) were combined peptide affinity greatly improved. A similar effect was observed for the mutations in peptide 45. Two of the individual mutations exhibited an affinity profile similar to that of peptide 45 (Table 2, peptides 45 (2), 61, and 63). When peptide 72 was synthesized bearing these two mutations, KD value of peptide 72 reached 27 nM, demonstrating a synergistic nature of these two mutations' interaction (Table 2, peptide 72). Analysis of the mutations found in peptide 33 revealed that Leucine 7 was vital for binding, as mutating this site lowered affinity (Table 2, compare peptides 9 (2) and 68). In contrast, ROM was not essential for binding, as mutating this site had no significant effect on the affinity (Table 2, compare peptides 9 (2), and 69). Further analysis of the individual mutations in peptide 27, showed that I13 is important for binding because peptide 67 that had I13C mutation showed no binding to IL-6. On the other hand, mutations C11K and H12V were mildly beneficial because peptides 65 and 66 had slightly better affinity than peptide 9 (2) (Table 2).

Analysis of combinations of mutations proved to be more challenging to interpret. For instance, when mutations L7I and R9M (originating from variant 33) were introduced into peptide 72, the KD value increased to 5 μM (Table 2, compare peptide 72 and 74). Conversely, when the same mutations were incorporated into peptides 38 and 45, the KD value remained around 100 nM (Table 2, peptides 51 and 58). Similarly, when R9M was combined with mutations present in peptides 45 and 72, affinity was not improved (Table 2, peptide 45 vs 75 and peptide 72 vs 80).

The influence of mutation I13C and the combination of mutations C11K and H12V, all originating from peptide 27, displayed a somewhat consistent pattern when integrated into different peptide sequences. Typically, they resulted in variants with much lower affinity than their parent sequences. For instance, when added to peptide 58, these mutations led to a notable increase in the KD value up to 1.5 μM (Table 2, peptides 76, 77 and 78). Likewise, when mutations C11K and H12V were introduced into peptide variant 51, its KD value increased from 128 nM to 563 nM (Table 2, peptides 51 and 83).

When all three mutations (C11K, H12V, and I13C) were combined, the results were mixed. For example, adding these mutations to peptide variant 51 lowered the KD value from 128 nM to 108 nM (Table 2, peptides 51 and 82). However, incorporating these mutations into peptide 58 increased the KD value from 71 nM to 247 nM (Table 2, peptide 79).

Similar confounding results were observed for the C11K mutation. When integrated into variant 58, it led to a decline in affinity, with KD increasing from 71 nM to 405 nM (Table 2, peptides 58 and 77).

In a final batch of combinations that were tested (Table 2, peptides 85-100), an effort was made to combine the most beneficial mutations from peptides 27, 33, 38 and 45. Surprisingly, only two good variants emerged (peptides 90 and 100). Both variants had a full set of mutations from peptide 27 and 33. Omission of any of these 6 mutations resulted in poor affinity which provides another example of synergy and not additivity between the residues.

### Example 4

### Controls

To evaluate the role of mutations in peptide 9 on its affinity a control peptide C1 was synthesized. This peptide is homologous to peptide 9, yet devoid of any mutations. In other words, it has two unmutated fragments of the IL-6R gene (FIG. 4). This peptide showed no binding to IL-6, demonstrating that mutations present in peptide 9 are critical for its interaction with IL-6 and emphasizes the utility of the degenerate sequences incorporated into the primers used during library construction. Without these intentionally designed mutations, active peptides would not have been discovered.

There are several publications describing peptides targeting IL-6 or its receptor. Su, J. L.; Lai, K. P.; Chen, C. A.; Yang, C. Y.; Chen, P. S.; Chang, C. C.; Chou, C. H.; Hu, C. L.; Kuo, M. L.; Hsieh, C. Y.; Wei, L. H. A Novel Peptide Specifically Binding to Interleukin-6 Receptor (Gp80) Inhibits Angiogenesis and Tumor Growth. Cancer Res 2005, 65 (11), 4827-4835; Yang, Z.; Feng, J.; Hu, M.; Li, Y.; Yu, M.; Qin, W.; Shen, B. A Novel HIL-6 Antagonist Peptide from Computer-Aided Design Contributes to Suppression of Apoptosis in M1 Cells. Biochem Biophys Res Commun 2004, 325 (2), 518-524. These peptides were designed rationally, selected from cDNA libraries by mRNA display or by a combination of molecular design and phage-display methods. However, none of these peptides made it to the clinic.

To figure out the reason for this failure, the inventors synthesized several of such peptides and compared to lead peptides of the invention (Table 2, control peptides). Most of the published peptides had no activity or a higher KD than the best peptides of the invention. For example, peptides CA11 and RA07 were described in Kobayashi, T.; Kakui, M.; Shibui, T.; Kitano, Y. In Vitro Selection of a Peptide Inhibitor of Human IL-6 Using MRNA Display. Mol Biotechnol 2011, 48 (2), 147-155. The inventors found that peptide CA11 had a KD of 50 μM and peptide RA07 did not bind IL-6 (Table 2, control peptides). Peptides PN-2529 and PN1974 were described in Ranganath, S.; Bhandari, A.; Avitahl-Curtis, N.; McMahon, J.; Wachtel, D.; Zhang, J.; Leitheiser, C.; Bernier, S. G.; Liu, G.; Tran, T. T.; Celino, H.; Tobin, J.; Jung, J.; Zhao, H.; Glen, K. E.; Graul, C.; Griffin, A.; Schairer, W. C.; Higgins, C.; Reza, T. L.; Mowe, E.; Rivers, S.; Scott, S.; Monreal, A.; Shea, C.; Bourne, G.; Coons, C.; Smith, A.; Tang, K.; Mandyam, R. A.; Masferrer, J.; Liu, D.; Patel, D. V; Fretzen, A.; Murphy, C. A.; Milne, G. T.; Smythe, M. L.; Carlson, K. E. Discovery and Characterization of a Potent Interleukin-6 Binding Peptide with Neutralizing Activity In Vivo. PLOS One 2015, 10 (11), e0141330. In a linear form, peptide PN-2519 showed no activity, while peptide PN-1974 had a KD of 61 nM.

Because unmodified peptides are prone to rapid degradation, the inventors protected the termini of peptide PN-1974 with acetylation and amidation, as the inventors did for all peptides of the invention. However, this eliminated its activity, (Table 2, peptide PN-1974 (2)). This direct comparison to the lead molecules of the invention suggests that the peptides of the invention have better chances to succeed as drug candidates.

### Example 5

### Assessment of Peptide Function

To verify the ability of the peptides of the invention to inhibit IL-6-signaling, they were tested with the IL-6 Bioassay Kit (Promega). This kit includes human cells engineered to express the IL-6 receptor and a luciferase reporter driven by a response element. When IL-6 binds, the IL-6R transduces intracellular signals resulting in luminescence (fluorescence). Inhibition of IL-6 interaction with the receptor leads to the decrease of the luciferase expression and lower fluorescence.

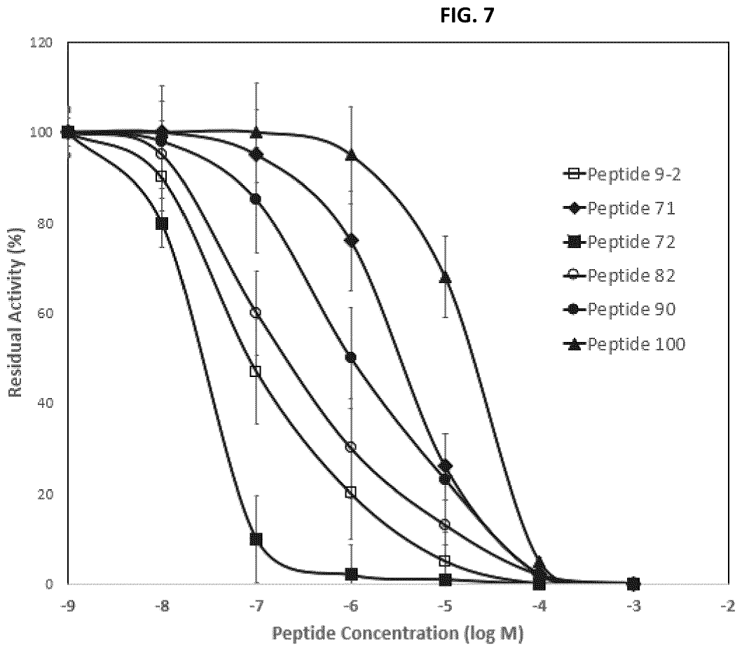

FIG. 7 shows a graph illustrating inhibition of IL-6-induced cell signaling by selected peptides. The selected peptides were incubated with IL-6 for 10 min before being added to cell culture medium. The observed IC50 values of the peptides were 85.43 nM for peptide 9 (2), 3.4 μM for peptide 71, 23.5 nM for peptide 72, 183 nM for peptide 82, 1.2 μM for peptide 90 and 16.8 μM for peptide 100. Peptides 27 (2), 38 (2) and 45 (2) were also tested. The IC50 for these peptides were 580 nM, 142 nM and 72 nM, respectively. In general, these results were consistent with the affinity data from the Biacore assay. The IC50 of peptide 38 (2) was 142 nM and that of peptide 45 (2) was 72 nM, which is similar to their parent peptide 9 (2) (IC50 85 nM).

However, peptides 90 and 100 which showed good affinity (KD of 30 nM and 50 nM respectively) did not perform well in the cell assay. Their IC50s were 1.2 μM and 16.8 μM respectively. This discrepancy is probably caused by the addition of the sulfur-containing residues (methionine and cysteines) which could be readily oxidized and are well known to cause chemical stability.

### Example 6

### Comparison of PepFusion Libraries to Other Methods

Non-specific contaminating sequences typically have no significant homology to the DNA sequence of the parent gene. In contrast, specific binders, such as peptide 9, are much more likely to align well with the parent molecule (FIG. 4). In case of peptide 9, its nucleotide identity to the parent sequence (IL-6R) is 80.5%. For this study, the minimum nucleotide identity for the PepFusion libraries was set arbitrarily at 66.7%.

The method described in this Example makes use of linear peptides, but the peptide libraries can be easily adapted for cyclic peptides by adding flanking cysteines as is known in the art. However, given that the inventors were able to identify a high affinity peptide using a linear peptide screen, peptide cyclization may be unnecessary.

### Constructs

Expression cassette was codon optimized, synthesized as gBlocks by IDT and cloned into the pBAD-HisA plasmid (Thermo Fisher Scientific). pBAD-HisA plasmid was amplified with primers P33 (AACTAAGCTTTTCCTCCTGTTAGCCCAAAAAAC) (SEQ ID NO: 98) and P34 (AATACTCGAGGCTGTTTTGGCGGATGAGAGAA) (SEQ ID NO: 99) introducing HindIII and XhoI restriction sites. Each PCR reaction (20 microliters) contained 20 ng of DNA template and 50 pmoles of each primer mixed with 10 microliters of Pfu Ultra II Hotstart 2× Mastermix (Agilent). The PCR reaction (20 microliters) was initially heated at 95° C. for 2.5 min followed by 30 cycles of denaturation at 94° C. for 15 sec., annealing at 55° C. for 15 sec. and extension at 72° C. for 6 min. Following amplification, the PCR fragment was gel-purified by the QIAGEN gel-band purification kit and digested with HindIII and XhoI restriction enzymes.

### Construction of Peptide Libraries

Two types of libraries were built. The first peptide library was random, built with 12 degenerate codons. This library was built with 12 NNK codons and amplified as two fragments which were united by ligation. The first fragment was amplified with flanking forward primer P23 (CCGCGAATGGTGAGATTGAGAA) (SEQ ID NO: 90) and the reverse primer T761 (MNNMNNMNNMNNMNNMNNACAaGAACcgagaccggatccCATTTAGCTGTCCTCCTT) (SEQ ID NO: 91). The second f fragment was amplified with primer T759 (NNKNNKNNKNNKNNKNNKTGTgGTTCtggtcttctgGGTAGCTTAGGTCACCATCACCA) (SEQ ID NO: 92) and the reverse primer P24 (ACGCAAAAAGGCCATCCGTCAG) (SEQ ID NO: 93).

The PCR reaction (20 microliters) was initially heated at 95° C. for 2.5 min followed by 30 cycles of denaturation at 94° C. for 15 sec., annealing at 55° C. for 15 sec. and extension at 72° C. for 40 sec. Following amplification, PCR fragments were gel-purified by the QIAGEN gel-band purification kit and mixed and ligated with T4 DNA ligase. The ligation reaction contained 20 microliters of 10× ligation buffer, 100 ng of fragment mix, 0.5 microliters of 100 mM ATP, 1 microliter of T4 DNA ligase (NEB cat #M0202S) and 1 microliter of T4 polynucleotide kinase. The reaction mix was incubated at room temperature and used as a template for PCR with flanking primers P311 (ATTATTttaattaaTTAATAGCCGGTGCCGTGGTGATGGTGATGGTGACCTA) (SEQ ID NO: 94) and P20 (ATTATTctcgagTTAATAGCCGGTGCCGTGGTGATGGTGATGGTGACCTA) (SEQ ID NO: 95), using program described above. The PCR fragment was gel-purified by the QIAGEN gel-band purification kit.

The second library was based on the published sequence of interleukin 6 receptor (IL-6R), specifically its IL-6-binding domain. Varghese, J. N.; Moritz, R. L.; Lou, M. Z.; Van Donkelaar, A.; Ji, H.; Ivancic, N.; Branson, K. M.; Hall, N. E.; Simpson, R. J. Structure of the Extracellular Domains of the Human Interleukin-6 Receptor Alpha-Chain. Proc Natl Acad Sci USA 2002, 99 (25), 15959-15964.

The method of library construction is presented in FIG. 3. The left fragment of the library was amplified with flanking forward primer P23 (CCGCGAATGGTGAGATTGAGAA) (SEQ ID NO: 90) and a mixture of the 432 of reverse primers, each containing an overhang of the 6 codons of the IR-6R binding domain. A degenerate nucleotide was inserted to the first or the second position of each codon. The right fragment was amplified with a mixture of the 432 forward primers, also containing overhangs of six degenerate codons of the IR-6R binding domain and the reverse primer P24 (ACGCAAAAAGGCCATCCGTCAG) (SEQ ID NO: 93). The PCR program was the same as described above.

### In Vitro Transcription

RNA was translated from amplified libraries using RiboMAX Large Scale RNA Production System T7 (Promega, Cat #P1300) according to the manufacturer's protocol and purified by RNeasy Mini Kit (Qiagen, Cat #74104)

Ligation of mRNA to DNA Linker with Puromycin

XL-PSO oligonucleotide was synthesized by IDT. The sequence of the oligonucleotide was: 5′-PsoC6-(uagccggug) 2′-OMc-AAAAAAAAAAAAAAA-Spacer9-Spaser9-ACC-Puro-3′ (SEQ ID NO: 96). To ligate this oligonucleotide to mRNA, the following reagents were mixed in a PCR tube: 29.5 microliters of RNAse-free water. 1 microliter of 1 M HEPES-KOH, pH 7.6, 5 microliters of 1 M KCl, 2 microliters of 25 mM spermidine, 0.5 microliters of 125 mM EDTA, 8 microliters of mRNA from previous step and 4 microliters of 100 mM of XL-PSO oligonucleotide. A PCR tube was placed in a PCR machine, heated to 70° C. for 5 min and cooled to 25° C. at 0.1° C./s speed. The mixture was then transferred to a 96-well plate on ice, put the 365-nm handheld UV lamp on top and irradiated the plate for 20 min. After that, cross-linked RNA was purified by RNeasy Mini Kit (Qiagen, Cat #74104).

### In Vitro Translation

Translation was performed using PUREexpress in vitro Protein Synthesis Kit (NEB, Cat #E6800S). The following reagents were mixed in a 1.5 ml tube: 20 microliters of Solution A, 15 microliters of solution B, 0.5 microliters of RNAsin Plus, 4.5 microliters of water and 10 microliters of cross-lined RNA (1 microgram/microliter). The mixture was incubated at 37° C. for 2 h.

Purification of Peptides with His-Tag

RNA-peptide complexes were purified using Ni-NTA magnetic beads (Qiagen, Cat #36111). 100 microliters of beads were washed with 300 microliters of wash buffer (50 mM NaH₂PO₄, 300 mM NaCl, 10 mM imidazole, 0.005% Tween 20), separated on the magnetic stand and suspended in 300 microliters of wash buffer. 25 microliters of RNA-peptide complexes from previous step were added to the washed beads and incubated on the end-over-end shaker for 30 min at room temperature. The beads were washed 3 times with the wash buffer followed by separation on the magnetic stand and eluted with 50 microliters of the elution buffer (50 mM NaH₂PO₄, 300 mM NaCl, 500 mM imidazole, 0.005% Tween 20).

Purification with Oligo-d (T) 25 Magnetic Beads

Oligo-d (T) 25 magnetic beads were purchased from New England Biolabs (Cat #S1419S). 100 microliters of the bead suspension was washed with 500 microliters of wash buffer I (20 mM Tris-HCl, pH 7.5, 500 mM NaCl, 1 mM EDTA) by suspending them in the buffer and separating on the magnetic stand followed by suspension in 50 microliters of wash buffer I. 50 microliters of the RNA-peptide complexes from the previous step were mixed with 50 microliters of binding buffer (100 mM Tris-HCl, pH 7.5, 1 M NaCl, 2 mM EDTA), heated at 65° C. for 2 min, placed on ice for 1 min and mixed with the washed beads. This mixture was incubated at room temperature for 5 min, then washed twice with 500 microliters of wash buffer I (20 mM Tris-HCl, pH 7.8, 500 mM NaCl, 1 mM EDTA) and once with 500 microliters of wash buffer II (20 mM Tris-HCl, pH 7.8, 200 mM NaCl, 1 mM EDTA).

### Affinity Selection

M-280 streptavidin magnetic from ThermoFischer Scientific (Cat #11206D) was used for affinity selection. Aliquots of 100 microliters of beads were dispensed into microcentrifuge tubes and washed with PBS twice using magnetic separation stand. An aliquot of biotinylated IL-6 (R&D Systems, cat #BT7270B) was added to the beads, incubated for 30 min at room temperature with rigorous shaking and washed with PBS 3 times using magnetic separation. Negative selection tubes were prepared in a similar way except no IL-6 was added.

Purified RNA-peptide complexes were first added to the negative selection tubes containing 200 microliters of PBS and incubated on a shaker at room temperature for 20 min. Next, tubes were placed on the magnetic separation stand and the supernatant was transferred to the positive selection tubes (with IL-6) and incubated on a shaker at room temperature for 30 min. Following incubation, wells were washed 3 times with PBS and one time with 4× diluted PBS. Bound molecules were eluted by solubilizing magnetic beads in 20 microliters of water and heating this mixture to 95° C. for 1 min. Following incubation, tubes were placed on a magnetic stand. The supernatant was transferred to a new tube and was used for cDNA synthesis and PCR.

### CDNA Synthesis and PCR

cDNA was synthesized using SuperScript III First-Strand Synthesis System (Invitrogen, Cat #18080-051). For each reaction, 2 microliters of 50 mM primer P19 (GTGGTGATGGTGATGGTGACCTA) (SEQ ID NO: 97) were mixed with 2 microliters of dNTP solution and 16 microliters of eluted RNA-peptide complexes and added to a PCR tube (ThermoFisher). This tube was then incubated at 65° C. for 5 min followed by cooling down to 4° C. for 1 min. 20 microliters mixture from the plate were transferred to a PCR tube and mixed with 20 microliters of the reaction mixture containing 4 microliters of 10× buffer, 8 microliters of 25 mM MgCl2, 4 microliters of 0.1 M DTT, 2 microliters of RNaseOUT and 2 microliters of Superscript III reverse transcriptase. This mixture was incubated at 50° C. for 50 min.

DNA corresponding to the strong binders surviving the selection was amplified with primers P17 and P20. Amplification was carried out using Taq DNA polymerase. The PCR reaction (20 microliters) was initially heated at 95° C. for 2.5 min, followed by 20 or 25 cycles of denaturation at 94° C. for 15 sec., annealing at 55° C. for 15 sec. and extension at 72° C. for 30 sec. Following amplification, PCR fragments were run on the agarose gel, and purified by the QIAGEN gel-band purification kit according to manufacturer's protocol.

### Sequence Analysis

Sequences of individual clones were analyzed by Next Generation Sequencing (NGS) and Sanger Sequencing. NGS was done by submitting PCR reactions from each selection cycle to GeneWiz for Amplicon-EZ service. For NGS, PCR products from each selection cycle were submitted to/were analyzed by GeneWiz for Amplicon-EZ service.

### The IL-6 Bioassay

The ability of peptides to inhibit IL-6 signaling pathway was tested with the IL-6 Bioassay from Promega (cat #JA2501). The bioluminescent signal is detected and quantified using Bio-Glo™ Luciferase Assay System. Different concentrations of the test peptides were mixed with IL-6 (0.5 nM) and added to the IL-6 Bioassay Cells according to the manufacturer's instructions. Cells were incubated for 6 hours in a humidified incubator at 37° C. and 5% CO₂ concentration. Following incubation, 75 microliters of the Bio-Glo™ Reagent was added to the cell culture and the luminescence was measured with a Biotek Synergy HTX plate reader. Data was analyzed according to manufacturer's recommendations.

### Biacore Assays

Binding reactions were performed in HBS-P/2% DMSO buffer (10 mM Hepes, pH 7.4, 150 mM NaCl, 0.05% P-20, 2% DMSO), which was filtered through 0.2 μM filters and degassed before use. Biotinylated recombinant human IL-6 (IL-6-Biotin) was bound to the surface of a Biacore SA sensor chip of a Biacore T200 surface plasmon resonance unit as follows. The streptavidin surface of the chip (flow cells 1 and 2) was activated by injecting 20 μl of 1 M NaCl/50 mM NaOH at 20 μl/min, in 3 pulses. Biotinylated Recombinant Human IL6 was diluted with HBS-P buffer and an aliquot of 100 μl (2 micrograms/ml) was injected into flow cell 2 at 5 microliters/min to yield a signal of 1400 response units (RU) (10 microliters were injected). Any remaining activated residues on the streptavidin surface of flow cells 1 and 2 were blocked with 30 microliters 500 nM free Biotin.

Affinity scouting assay was performed at a flow rate of 30 microliters/min at 25° C. The analyte (60 microliter aliquots of peptides, at concentrations of 20 μM in HBS-P/2% DMSO buffer) was injected into flow cells 1 and 2 and the association reaction was recorded. The surface was then washed with HBS-P/2% DMSO for 3 min and the dissociation of the analyte from the bound IL6 was followed over time. The surfaces of the flow cells were regenerated with 30 microliters of 10 mM NaOH, followed 50 microliters of HBS-P/2% DMSO buffer.

Kinetics analysis of binding was performed at a flow rate of 30 microliters/min at 25° C. The analyte (60 microliter aliquots of peptides, at concentrations of 0, 1.25 μM, 2.5 μM, 5 μM, 10 μM and 20 μM in HBS-P/2% DMSO buffer) was injected into flow cells 1 and 2 and the association reaction was recorded. The surface was then washed with HBS-P/2% DMSO for 3 min and the dissociation of the analyte from the bound IL6 was followed over time. The surfaces of the flow cells were regenerated with 30 microliters of 10 mM NaOH, followed 50 microliters of HBS-P/2% DMSO buffer. Sensorgrams were analyzed using BIAeveluation 3.2.1 software (Cytiva). Values from the reference flow cell were subtracted to obtain the values for specific binding. Data were globally fitted to the Langmuir model for a 1:1 mass transfer binding.

### Peptide Synthesis.

Peptides were synthesized by Elim or GL Biochem (Shanghai) Ltd.

While the following description details the preferred embodiments of the present disclosure, it is to be understood that the invention is not limited in its application to the details of compositions or steps of methods illustrated in the accompanying figures, since the compositions and methods are capable of other embodiments and of being practiced in various ways. Additionally, the disclosure shows and describes only the preferred embodiments but as mentioned above, it is to be understood that the preferred embodiments are capable of being formed in various other combinations, modifications, and environments and are capable of changes or modifications within the scope of the invention concepts as expressed herein, commensurate with the above teachings and/or the skill or knowledge of the relevant art. The embodiments described herein above are further intended to explain the best modes known by applicant and to enable others skilled in the art to utilize the disclosure in such, or other, embodiments and with the various modifications required by the particular application or uses thereof. Accordingly, the description is not intended to limit the invention to the form disclosed herein. Also, it is intended that the appended claims be construed to include alternative embodiments. It will be further understood that various changes in the details, materials, and arrangements of the parts which have been described and illustrated above to explain the nature of this invention may be made by those skilled in the art without departing from the principle and scope of the invention as recited in the following claims.

What is claimed is:

1. A method of constructing a peptide library, comprising:
1) identifying a protein of interest;
2) synthesizing DNA primers comprising DNA sequences, wherein the DNA sequences are determined by the following steps:
a) writing a DNA sequence that codes for the identified protein or a segment thereof;
b) splitting the written DNA sequence of the identified protein or segment thereof into a plurality of written DNA sequences from 3 to 18 nucleotides or longer, thereby forming a list of short written DNA sequences;
c) introducing degeneracy into one or more of the short written DNA sequence from step (b) to create one or more short degenerate written DNA sequences; and
d) adding the one or more short degenerate written DNA sequences to written DNA primer sequences designed to amplify a DNA fragment of a screening construct to create written DNA primer sequences with overhangs; and
e) synthesizing the written DNA primer sequences with overhangs from step (d) to create synthesized primers;
3) amplifying DNA fragments using at least three or more of the synthesized primers from step (e) and one or more DNA polymerases;
4) mixing, phosphorylating and ligating the amplified DNA fragments from step (3), thereby forming a DNA library; and
5) expressing peptides coded by the DNA library formed in step (4) in an appropriate system in vitro or in vivo, thereby forming a first peptide library having peptides with a pharmacologic activity.

2. The method of claim 1, wherein in step (c), degeneracy is introduced into the one or more short written DNA sequences by one of the following steps:
changing the nucleotide at the first position of every codon;
changing the nucleotide at the second position of every codon;
changing the nucleotide at the first position of the first codon, the second position of the second codon and repeating this pattern for the entire short written DNA sequence;
changing the second position of the first codon and the first position of the second codon and repeating this pattern for the entire short written DNA sequence; and
changing the first position and/or the second position of less than every codon.

3. The method of claim 1, wherein the segment of the identified protein is a binding domain.

4. The method of claim 1, wherein steps (2) through (5) are repeated, thereby forming a second peptide library with an improved pharmacologic activity, compared to the first peptide library.

5. The method of claim 1, wherein the segment of the identified protein consists of loops and/or secondary protein structures.

6. The method of claim 3, wherein the size of the binding domain is at least 220 or more amino acids.

7. The method of claim 1, wherein in step 3, the primers are used simultaneously.

8. The method of claim 1, wherein in step 3, one or more of the synthesized primers are designed for a segment of the binding domain, wherein the segment comprises 10 or more amino acids.

9. The method of claim 1, wherein in step 3, all of the synthesized primers are designed for a segment of the binding domain, wherein the segment comprises 10 or more amino acids.

10. The method of claim 1, wherein amplified DNA fragments from step 3 are digested by type IIS restriction enzymes.

11. The method of claim 1, wherein ligated DNA fragments from step 4 are amplified with the flanking primers.

12. The method of claim 1, wherein each DNA sequence in step (c) is different from the DNA sequence from step (b) by only one codon.

13. The method of claim 1, wherein the degenerate written short DNA sequences in step 3 are linked as overhangs to primers with one forward primer and one reverse primer.

14. The method of claim 1, wherein the one or more DNA polymerases in step (e) comprise a Vent polymerase generating blunt DNA ends.

15. The method of claim 1, further comprising the following steps:
6) selecting and synthesizing peptides from the first peptide library;
7) measuring an affinity of binding of the selected and synthesized peptides from the first library to the desired biological target; and
8) measuring efficacy of the selected and synthesized peptides from the first library in vitro and/or in vivo.

16. The method of claim 4, further comprising the following steps:
6) selecting and synthesizing peptides from the second peptide library;
7) measuring an affinity of binding of the selected and synthesized peptides from the second library to the desired biological target; and
8) measuring efficacy of the selected and synthesized peptides from the second library in vitro and/or in vivo.

17. The method of claim 16, wherein the desired biological target is interleukin-6.

18. A peptide from a peptide library produced by the method of claim 16, the peptide comprising the amino acid sequence as set forth in any one of SEQ ID Nos: 54, 55, 70, 73, and 83, or an amido acid sequence having more than 70% homology thereto.

19. The peptide of claim 18, wherein the amino acid sequence has more than 75% homology to any one of SEQ ID Nos: 34, 35, 54, 55, 70, 73, and 83.

20. The peptide of claim 18, wherein the amino acid sequence has more than 80% homology to any one of SEQ ID Nos: 34, 35 54, 55, 70, 73, and 83.

21. The peptide of claim 18, wherein the amino acid sequence has more than 85% homology to any one of SEQ ID Nos: 34, 35, 54, 55, 70, 73, and 83.

22. The peptide of claim 18, wherein the amino acid sequence has more than 90% homology to any one of SEQ ID Nos: 34, 35, 54, 55, 70, 73, and 83.

23. The peptide of claim 18, wherein the amino acid sequence has more than 95% homology to any one of SEQ ID Nos: 34, 35, 54, 55, 70, 73, and 83.

24. The peptide of claim 18, wherein the peptide is SEQ ID No: 55 or an amido acid sequence having more than 70% homology thereto.

25. The peptide of claim 24, wherein the amino acid sequence has more than 75% homology thereto.

26. The peptide of claim 24, wherein the amino acid sequence has more than 80% homology thereto.

27. The peptide of claim 24, wherein the amino acid sequence has more than 85% homology thereto.

28. The peptide of claim 24, wherein the amino acid sequence has more than 90% homology thereto.

29. The peptide of claim 24, wherein the amino acid sequence has more than 95% homology thereto.

30. The peptide of claim 18, wherein the peptide's termini are protected.

31. The peptide of claim 18, wherein the N terminus of the peptide is acetylated and the C terminus is amidated.

32. A peptide which exhibits antagonistic activity directed against interleukin-6, the peptide comprising the amino acid sequence as set forth in any one of SEQ ID Nos: 34, 35, 54, 55, 70, 73, and 83, or an amido acid sequence having more than 70% homology thereto.

33. A method of inhibiting IL-6 signaling in a cell, the method comprising contacting a cell with an effective amount of the peptide according to claim 18.

34. A method of treating a disease or condition mediated by IL-6 mechanism comprising administering to a patient in need thereof an effective amount of the peptide according to claim 18.

35. The method of claim 34, wherein the disease or condition comprises an inflammatory, degenerative or an autoimmune disease or condition.

36. The method of claim 34, wherein the disease or condition is selected from the group consisting of rheumatoid arthritis, Crohn's disease, Castleman disease, systemic lupus erythematosus, juvenile idiopathic arthritis, giant cell arteritis, ulcerative colitis, psoriatic arthritis, ankylosing spondylitis, multiple myeloma, systemic sclerosis, Still's disease, severe acute respiratory syndrome coronavirus 2 (SARS-CoV-2) infection (COVID-19), chronic obstructive pulmonary disease, atherosclerosis, osteoporosis, type 2 diabetes mellitus, depression, Alzheimer's disease, and cytokine release syndrome.


SEQUENCE LISTING (99 sequences)

SEQ ID NO: 1 - protein, 14 aa, synthetic construct
SVRDLLRRMCHIVG

SEQ ID NO: 2 - protein, 14 aa, synthetic construct
SVRIYLRRMCHIVG

SEQ ID NO: 3 - protein, 14 aa, synthetic construct
SVRDLLNRICHIVG

SEQ ID NO: 4 - protein, 14 aa, synthetic construct
SVRDPHNRMCHIVG

SEQ ID NO: 5 - protein, 14 aa, synthetic construct
SVRDLLRMFCHIVG

SEQ ID NO: 6 - protein, 15 aa, synthetic construct
ETIRDLLRRMCHIVG

SEQ ID NO: 7 - protein, 14 aa, synthetic construct
SVRDLHKMMCHIVG

SEQ ID NO: 8 - protein, 15 aa, synthetic construct
LEIRDLLRRMCHIVG

SEQ ID NO: 9 - protein, 14 aa, synthetic construct
SVRDLLRRMKVCVG

SEQ ID NO: 10 - protein, 14 aa, synthetic construct
ACMDLLRRMCHIVG

SEQ ID NO: 11 - protein, 15 aa, synthetic construct
LGHRDLLRRMCHIVG

SEQ ID NO: 12 - protein, 14 aa, synthetic construct
SVRDNFHRMCHIVG

SEQ ID NO: 13 - protein, 14 aa, synthetic construct
SVRDLPRIMCHIVG

SEQ ID NO: 14 - protein, 14 aa, synthetic construct
SVSIHLRRMCHIVG

SEQ ID NO: 15 - protein, 14 aa, synthetic construct
SVRDLIRMMCHIVG

SEQ ID NO: 16 - protein, 14 aa, synthetic construct
SVHIHLRRMCHIVG

SEQ ID NO: 17 - protein, 15 aa, synthetic construct
CVYRDLLRRMCHIVG

SEQ ID NO: 18 - protein, 14 aa, synthetic construct
SNYTLLRRMCHIVG

SEQ ID NO: 19 - protein, 14 aa, synthetic construct
SVRDLPTQMCHIVG

SEQ ID NO: 20 - protein, 15 aa, synthetic construct
ATIRDLLRRMCHIVG

SEQ ID NO: 21 - protein, 14 aa, synthetic construct
SVRDANYRMCHIVG

SEQ ID NO: 22 - protein, 14 aa, synthetic construct
SVRDLLHGRCHIVG

SEQ ID NO: 23 - protein, 15 aa, synthetic construct
NFRRDLLRRMCHIVG

SEQ ID NO: 24 - protein, 15 aa, synthetic construct
GNSRDLLRRMCHIVG

SEQ ID NO: 25 - protein, 14 aa, synthetic construct
SVRDLLSHNCHIVG

SEQ ID NO: 26 - protein, 14 aa, synthetic construct
SVRDLQKQMCHIVG

SEQ ID NO: 27 - protein, 15 aa, synthetic construct
FAFRDLLRRMCHIVG

SEQ ID NO: 28 - protein, 15 aa, synthetic construct
SVRDLLRRMCHPRLG

SEQ ID NO: 29 - protein, 14 aa, synthetic construct
SVRDLQSTMCHIVG

SEQ ID NO: 30 - protein, 14 aa, synthetic construct
SVRDWTPRMCHIVG

SEQ ID NO: 31 - protein, 14 aa, synthetic construct
SVRDLLRRMCVFWG

SEQ ID NO: 32 - protein, 14 aa, synthetic construct
  SITE 1: note = Acetylated residue
  SITE 14: note = Amidated residue
SVRDLLRRMCHIVG

SEQ ID NO: 33 - protein, 14 aa, synthetic construct
  SITE 1: note = Acetylated residue
  SITE 14: note = Amidated residue
SVRDLLRRMKVCVG

SEQ ID NO: 34 - protein, 15 aa, synthetic construct
  SITE 1: note = Acetylated residue
  SITE 15: note = Amidated residue
ATIRDLLRRMCHIVG

SEQ ID NO: 35 - protein, 15 aa, synthetic construct
  SITE 1: note = Acetylated residue
  SITE 15: note = Amidated residue
FAFRDLLRRMCHIVG

SEQ ID NO: 36 - protein, 15 aa, synthetic construct
  SITE 1: note = Acetylated residue
  SITE 15: note = Amidated residue
ATIRDLIRMMCHIVG

SEQ ID NO: 37 - protein, 15 aa, synthetic construct
  SITE 1: note = Acetylated residue
  SITE 15: note = Amidated residue
ATIRDLLSHNCHIVG

SEQ ID NO: 38 - protein, 15 aa, synthetic construct
  SITE 1: note = Acetylated residue
  SITE 15: note = Amidated residue
ATIRDLLRRMKVCVG

SEQ ID NO: 39 - protein, 15 aa, synthetic construct
  SITE 1: note = Acetylated residue
  SITE 15: note = Amidated residue
ASVRDLLRRMCHIVG

SEQ ID NO: 40 - protein, 14 aa, synthetic construct
  SITE 1: note = Acetylated residue
  SITE 14: note = Amidated residue
TVRDLLRRMCHIVG

SEQ ID NO: 41 - protein, 14 aa, synthetic construct
  SITE 1: note = Acetylated residue
  SITE 14: note = Amidated residue
SIRDLLRRMCHIVG

SEQ ID NO: 42 - protein, 15 aa, synthetic construct
  SITE 1: note = Acetylated residue
  SITE 15: note = Amidated residue
FAFRDLIRMMCHIVG

SEQ ID NO: 43 - protein, 15 aa, synthetic construct
  SITE 1: note = Acetylated residue
  SITE 15: note = Amidated residue
FAFRDLLSHNCHIVG

SEQ ID NO: 44 - protein, 15 aa, synthetic construct
  SITE 1: note = Acetylated residue
  SITE 15: note = Amidated residue
FAFRDLLRRMKVCVG

SEQ ID NO: 45 - protein, 15 aa, synthetic construct
  SITE 1: note = Acetylated residue
  SITE 15: note = Amidated residue
FSVRDLLRRMCHIVG

SEQ ID NO: 46 - protein, 14 aa, synthetic construct
  SITE 1: note = Acetylated residue
  SITE 14: note = Amidated residue
AVRDLLRRMCHIVG

SEQ ID NO: 47 - protein, 14 aa, synthetic construct
  SITE 1: note = Acetylated residue
  SITE 14: note = Amidated residue
SFRDLLRRMCHIVG

SEQ ID NO: 48 - protein, 14 aa, synthetic construct
  SITE 1: note = Acetylated residue
  SITE 14: note = Amidated residue
SVRDLIRMMKVCVG

SEQ ID NO: 49 - protein, 14 aa, synthetic construct
  SITE 1: note = Acetylated residue
  SITE 14: note = Amidated residue
SVRDLLRRMKHIVG

SEQ ID NO: 50 - protein, 14 aa, synthetic construct
  SITE 1: note = Acetylated residue
  SITE 14: note = Amidated residue
SVRDLLRRMCVIVG

SEQ ID NO: 51 - protein, 14 aa, synthetic construct
  SITE 1: note = Acetylated residue
  SITE 14: note = Amidated residue
SVRDLLRRMCHCVG

SEQ ID NO: 52 - protein, 14 aa, synthetic construct
  SITE 1: note = Acetylated residue
  SITE 14: note = Amidated residue
SVRDLIRRMCHIVG

SEQ ID NO: 53 - protein, 14 aa, synthetic construct
  SITE 1: note = Acetylated residue
  SITE 14: note = Amidated residue
SVRDLLRMMCHIVG

SEQ ID NO: 54 - protein, 14 aa, synthetic construct
  SITE 1: note = Acetylated residue
  SITE 14: note = Amidated residue
TIRDLLRRMCHIVG

SEQ ID NO: 55 - protein, 15 aa, synthetic construct
  SITE 1: note = Acetylated residue
  SITE 15: note = Amidated residue
FSFRDLLRRMCHIVG

SEQ ID NO: 56 - protein, 14 aa, synthetic construct
  SITE 1: note = Acetylated residue
  SITE 14: note = Amidated residue
SVRDLLRRMKVIVG

SEQ ID NO: 57 - protein, 15 aa, synthetic construct
  SITE 1: note = Acetylated residue
  SITE 15: note = Amidated residue
FSFRDLIRMMCHIVG

SEQ ID NO: 58 - protein, 15 aa, synthetic construct
  SITE 1: note = Acetylated residue
  SITE 15: note = Amidated residue
FAFRDLLRMMCHIVG

SEQ ID NO: 59 - protein, 15 aa, synthetic construct
  SITE 1: note = Acetylated residue
  SITE 15: note = Amidated residue
FAFRDLIRMMKVIVG

SEQ ID NO: 60 - protein, 15 aa, synthetic construct
  SITE 1: note = Acetylated residue
  SITE 15: note = Amidated residue
FAFRDLIRMMKHIVG

SEQ ID NO: 61 - protein, 15 aa, synthetic construct
  SITE 1: note = Acetylated residue
  SITE 15: note = Amidated residue
FAFRDLIRMMCVIVG

SEQ ID NO: 62 - protein, 15 aa, synthetic construct
  SITE 1: note = Acetylated residue
  SITE 15: note = Amidated residue
FAFRDLIRMMKVCVG

SEQ ID NO: 63 - protein, 15 aa, synthetic construct
  SITE 1: note = Acetylated residue
  SITE 15: note = Amidated residue
FSFRDLLRMMCHIVG

SEQ ID NO: 64 - protein, 15 aa, synthetic construct
  SITE 1: note = Acetylated residue
  SITE 15: note = Amidated residue
FSFRDLLRMMKVIVG

SEQ ID NO: 65 - protein, 15 aa, synthetic construct
  SITE 1: note = Acetylated residue
  SITE 15: note = Amidated residue
ATIRDLIRMMKVCVG

SEQ ID NO: 66 - protein, 15 aa, synthetic construct
  SITE 1: note = Acetylated residue
  SITE 15: note = Amidated residue
ATIRDLIRMMKVIVG

SEQ ID NO: 67 - protein, 14 aa, synthetic construct
  SITE 1: note = Acetylated residue
  SITE 14: note = Amidated residue
TIRDLLRMMKVIVG

SEQ ID NO: 68 - protein, 15 aa, synthetic construct
  SITE 1: note = Acetylated residue
  SITE 15: note = Amidated residue
FSVRDLLRRMKHIVG

SEQ ID NO: 69 - protein, 15 aa, synthetic construct
  SITE 1: note = Acetylated residue
  SITE 15: note = Amidated residue
FSVRDLLRMMKHIVG

SEQ ID NO: 70 - protein, 15 aa, synthetic construct
  SITE 1: note = Acetylated residue
  SITE 15: note = Amidated residue
FSVRDLIRMMKVCVG

SEQ ID NO: 71 - protein, 14 aa, synthetic construct
  SITE 1: note = Acetylated residue
  SITE 14: note = Amidated residue
SFRDLLRRMKHIVG

SEQ ID NO: 72 - protein, 14 aa, synthetic construct
  SITE 1: note = Acetylated residue
  SITE 14: note = Amidated residue
SFRDLLRMMKHIVG

SEQ ID NO: 73 - protein, 14 aa, synthetic construct
  SITE 1: note = Acetylated residue
  SITE 14: note = Amidated residue
SFRDLIRMMKVCVG

SEQ ID NO: 74 - protein, 15 aa, synthetic construct
  SITE 1: note = Acetylated residue
  SITE 15: note = Amidated residue
FSFRDLLRRMKHIVG

SEQ ID NO: 75 - protein, 15 aa, synthetic construct
  SITE 1: note = Acetylated residue
  SITE 15: note = Amidated residue
FSFRDLLRMMKHIVG

SEQ ID NO: 76 - protein, 15 aa, synthetic construct
  SITE 1: note = Acetylated residue
  SITE 15: note = Amidated residue
FSFRDLIRMMKVCVG

SEQ ID NO: 77 - protein, 15 aa, synthetic construct
  SITE 1: note = Acetylated residue
  SITE 15: note = Amidated residue
ATIRDLLRRMKHIVG

SEQ ID NO: 78 - protein, 15 aa, synthetic construct
  SITE 1: note = Acetylated residue
  SITE 15: note = Amidated residue
ATIRDLLRMMKHIVG

SEQ ID NO: 79 - protein, 15 aa, synthetic construct
  SITE 1: note = Acetylated residue
  SITE 15: note = Amidated residue
ATIRDLIRMMKHIVG

SEQ ID NO: 80 - protein, 14 aa, synthetic construct
  SITE 1: note = Acetylated residue
  SITE 14: note = Amidated residue
TIRDLLRRMKHIVG

SEQ ID NO: 81 - protein, 14 aa, synthetic construct
  SITE 1: note = Acetylated residue
  SITE 14: note = Amidated residue
TIRDLLRMMKHIVG

SEQ ID NO: 82 - protein, 14 aa, synthetic construct
  SITE 1: note = Acetylated residue
  SITE 14: note = Amidated residue
TIRDLIRMMKHIVG

SEQ ID NO: 83 - protein, 14 aa, synthetic construct
  SITE 1: note = Acetylated residue
  SITE 14: note = Amidated residue
TIRDLIRMMKVCVG

SEQ ID NO: 84 - protein, 19 aa, synthetic construct
NQQLIEEIIQILHKIFEIL

SEQ ID NO: 85 - protein, 19 aa, synthetic construct
INTLLSEINSILLDIISLL

SEQ ID NO: 86 - protein, 21 aa, synthetic construct
  SITE 3: note = Cyclohexyl-L-alanine
  SITE 9: note = 4-fluoro-L-phenylalanine
  SITE 17: note = Gamma-carboxyglutamic acid
FDXDIHLLXLPTEWEKXKNEE

SEQ ID NO: 87 - protein, 21 aa, synthetic construct
  SITE 3: note = Homoleucine
FDXDIHLLFLPTEWEKDKNEE

SEQ ID NO: 88 - protein, 21 aa, synthetic construct
  SITE 1: note = Acetylated residue
  SITE 3: note = Homoleucine
  SITE 21: note = Amidated residue
FDXDIHLLFLPTEWEKDKNEE

SEQ ID NO: 89 - protein, 14 aa, synthetic construct
SVKDLQHGLRHVVG

SEQ ID NO: 90 - DNA, 22 bp, synthetic construct
  source 1..22: mol_type = other DNA
ccgcgaatggtgagattgagaa

SEQ ID NO: 91 - DNA, 57 bp, synthetic construct
  source 1..57: mol_type = other DNA
  misc_difference 2: note = a, c, t, g, unknown or other
  misc_difference 3: note = a, c, t, g, unknown or other
  misc_difference 5: note = a, c, t, g, unknown or other
  misc_difference 6: note = a, c, t, g, unknown or other
  misc_difference 8: note = a, c, t, g, unknown or other
  misc_difference 9: note = a, c, t, g, unknown or other
  misc_difference 11: note = a, c, t, g, unknown or other
  misc_difference 12: note = a, c, t, g, unknown or other
  misc_difference 14: note = a, c, t, g, unknown or other
  misc_difference 15: note = a, c, t, g, unknown or other
  misc_difference 17: note = a, c, t, g, unknown or other
  misc_difference 18: note = a, c, t, g, unknown or other
mnnmnnmnnmnnmnnmnnacaagaaccgagaccggatcccatttagctgtcctcctt

SEQ ID NO: 92 - DNA, 59 bp, synthetic construct
  source 1..59: mol_type = other DNA
  misc_difference 1: note = a, c, t, g, unknown or other
  misc_difference 2: note = a, c, t, g, unknown or other
  misc_difference 4: note = a, c, t, g, unknown or other
  misc_difference 5: note = a, c, t, g, unknown or other
  misc_difference 7: note = a, c, t, g, unknown or other
  misc_difference 8: note = a, c, t, g, unknown or other
  misc_difference 10: note = a, c, t, g, unknown or other
  misc_difference 11: note = a, c, t, g, unknown or other
  misc_difference 13: note = a, c, t, g, unknown or other
  misc_difference 14: note = a, c, t, g, unknown or other
  misc_difference 16: note = a, c, t, g, unknown or other
  misc_difference 17: note = a, c, t, g, unknown or other
nnknnknnknnknnknnktgtggttctggtcttctgggtagcttaggtcaccatcacca

SEQ ID NO: 93 - DNA, 22 bp, synthetic construct
  source 1..22: mol_type = other DNA
acgcaaaaaggccatccgtcag

SEQ ID NO: 94 - DNA, 52 bp, synthetic construct
  source 1..52: mol_type = other DNA
attattttaattaattaatagccggtgccgtggtgatggtgatggtgaccta

SEQ ID NO: 95 - DNA, 50 bp, synthetic construct
  source 1..50: mol_type = other DNA
attattctcgagttaatagccggtgccgtggtgatggtgatggtgaccta

SEQ ID NO: 96 - RNA, 25 nt, synthetic construct
  source 1..25: mol_type = other RNA
  modified_base 1: mod_base = OTHER; note = Nucleotide attached via a C6 alkyl chain to a psoralen moiety
  modified_base 10: mod_base = OTHER; note = O2'-Methylycytidine-5'-monophosphate
  modified_base 25: mod_base = OTHER; note = Nucleotide attached to another sequence via 2x 9-O-Dimethoxytrityl-triethylene glycol,1-[(2-cyanoethyl)-( N,N-diisopropyl)]-phosphoramidite spacers
tagccggtgcaaaaaaaaaaaaaaa

SEQ ID NO: 97 - DNA, 23 bp, synthetic construct
  source 1..23: mol_type = other DNA
gtggtgatggtgatggtgaccta

SEQ ID NO: 98 - DNA, 33 bp, synthetic construct
  source 1..33: mol_type = other DNA
aactaagcttttcctcctgttagcccaaaaaac

SEQ ID NO: 99 - DNA, 32 bp, synthetic construct
  source 1..32: mol_type = other DNA
aatactcgaggctgttttggcggatgagagaa